(19) United States
(12) Patent Application Publication
(10) Pub. No.: US 2016/0266083 A1
(43) Pub. Date: Sep. 15, 2016

(54) METHOD AND SYSTEM FOR DETECTING ALLERGENS IN A CONSUMABLE

(71) Applicant: 6SensorLabs, Inc., San Francisco, CA (US)

(72) Inventors: Jingqing Zhang, San Francisco, CA (US); Scott Sundvor, San Francisco, CA (US); Jack Shirajian, San Francisco, CA (US)

(21) Appl. No.: 15/065,198

(22) Filed: Mar. 9, 2016

Related U.S. Application Data
(60) Provisional application No. 62/130,268, filed on Mar. 9, 2015.
(60) Provisional application No. 62/130,295, filed on Mar. 9, 2015.

(51) Int. Cl. G01N 33/02; G01N 33/53

(52) U.S. Cl. CPC G01N 33/02; G01N 33/5306

(57) ABSTRACT

An embodiment of a method for detecting a target substance in a consumable sample comprises receiving a consumable sample; extracting a test sample including a target of interest from the consumable sample with an extraction buffer; transmitting the test sample to an immunoassay device; generating a first indication characteristic of presence of the target substance in the consumable sample upon interfacing the test sample with a probe configured to react with the allergen constituent at the immunoassay device; generating a second indication characteristic upon interfacing the test sample with a bioconjugate configured to react with the allergen constituent at the immunoassay device; generating an analysis of the indication, including at least one of a presence of and an amount of the target substance present in the consumable sample; and providing information derived from the analysis to a user associated with the consumable sample.

### CROSS-REFERENCE TO RELATED APPLICATIONS

This application claims the benefit of U.S. Provisional Application No. 62/130,268 filed 9 Mar. 2015 and U.S. Provisional Application No. 62/130,295 filed 9 Mar. 2015, which are each incorporated in its entirety by this reference.

### TECHNICAL FIELD

This invention relates generally to the consumer assay device field, and more specifically to an improved method and system for detecting allergens in a consumable.

### BACKGROUND

A wide variety of consumables (e.g., foods, beverage, cosmetics, etc.) contain contaminants, toxins, allergens, and/or other substances that are of interest to all or specific types of consumers. In recent years, an increase in the number of consumers with an identified allergy (e.g., gluten allergy, dairy allergy, fish allergy, nut allergy, soy allergy, cosmetic allergy, etc.) has contributed to a number of products that omit ingredients having an associated allergen; however, such consumers are still at risk for consuming items with a harmful substance when the items do not have adequate labeling or documentation. Various systems and methods exist for detection of toxins and harmful substances present in a sample; however, current methods and systems are deficient due to one or more of: a time-intensive manner of receiving test results, a labor-intensive manner of receiving test results, a non-automated manner of processing samples, system bulk, system non-portability, and other factors that contribute to inconveniencing a consumer using such systems.

Due to these and other defects of current methods and systems for detecting substances in consumables, there is thus a need for an improved method and system for detecting such substances. This invention provides such a method and system.

### BRIEF DESCRIPTION OF THE FIGURES

FIGS. 1A and 1B depict embodiment of a method for detecting allergens in a consumable;

FIG. 2 depicts an example of an allergen indicator module in an embodiment of a method for detecting allergens in a consumable;

FIG. 3 depicts examples of signal generation in an embodiment of a method for detecting allergens in a consumable;

FIGS. 4A and 4B depict examples of an analysis step in an embodiment of a method for detecting allergens in a consumable;

FIGS. 5 and 6 depict variations of a portion of an embodiment of a method for detecting allergens in a consumable; and

FIGS. 7A-7C examples of a portion of an embodiment of a method for detecting allergens in a consumable.

### DESCRIPTION OF THE PREFERRED EMBODIMENTS

The following description of preferred embodiments of the invention is not intended to limit the invention to these preferred embodiments, but rather to enable any person skilled in the art to make and use this invention.

### 1. Overview

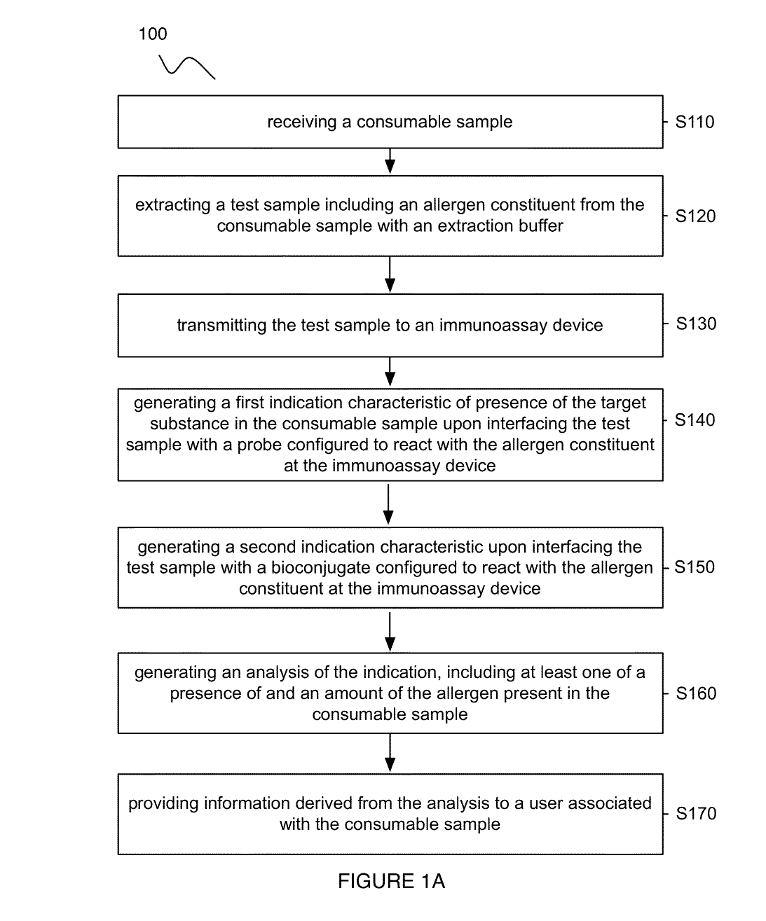

As shown in FIGS. 1A and 1B, an embodiment of a method 100 for detecting a target substance in a consumable sample comprises: receiving a consumable sample S110; extracting a test sample including an allergen constituent from the consumable sample with an extraction buffer S120; transmitting the test sample to an immunoassay device S130; generating a first indication characteristic of presence of the target substance in the consumable sample upon interfacing the test sample with a probe configured to react with the allergen constituent at the immunoassay device S140; generating a second indication characteristic upon interfacing the test sample with a bioconjugate configured to react with the allergen constituent at the immunoassay device S150; generating an analysis of the indication, including at least one of a presence of and an amount of the target substance present in the consumable sample S160; and providing information derived from the analysis to a user associated with the consumable sample S170.

The method 100 functions to receive and process a sample of a consumable (e.g., food, beverage, cosmetic, etc.) in order to enable detection of one or more substances of interest within the sample. In examples, the substances can include any one or more of: an allergen (e.g., gluten allergen, a dairy-derived allergen, a nut allergen, a fish allergen, an egg-derived allergen, a soy derived allergen, a peanut-derived allergen, shellfish-derived allergens, etc.), a toxin, a bacterium, a fungus, a pesticide, a heavy metal, a chemical or biological compound (e.g., a fat, a protein, a sugar, a salt, etc.), and any other suitable substance of interest. The method 100 is preferably configured to enable rapid detection of one or more allergens in a consumable sample, in a facile manner that is time-efficient, cost-efficient, labor efficient, etc., for the entity interested in determining presence of the substance(s) within the consumable sample. As such, the method 100 is preferably configured to automatically or semi-automatically process the sample in a manner that is non-burdensome to the consumer, and to provide information quickly regarding presence of the harmful substance(s) within the sample.

### 2. Benefits

The method 100 can provide significant improvements over limitations existing in current methods for allergen testing (e.g., mass spectroscopy, PCR techniques, standard ELISA, etc.), which are expensive, not currently suitable for consumer use, involve many processing steps, are unable to detect target proteins (that can cause an allergic response), and/or have limited accuracy. In a specific application, the method 100 can provide an indication and/or an analysis of presence of gluten in a food sample on the order of minutes (e.g., 1-5 minutes), using an improved allergen extraction process, streamlined and automatic sample processing, and an improved allergen indicator module comprising a test strip. However, variations of the specific application can alternatively involve detection of any other suitable type or number of allergens (e.g., gluten allergen, a dairy-derived allergen, a nut allergen, a fish allergen, an egg-derived allergen, a soy derived allergen, a peanut-derived allergen, shellfish-derived allergens, etc.) or any other substances of interest in a consumable sample, within any other suitable time frame, and using any other suitable substance indicator module. The method 100 is preferably implemented at least in part by a portion of the system 100 described in U.S. application Ser. No. 14/498,298, filed on 26 Sep. 2014 and entitled “System and Method for Detection of Target Substance”, which is herein incorporated in its entirety by this reference; however, the method 100 can alternatively be implemented using any other suitable system.

### 3. Method.

### 3.1 Receiving a Consumable Sample.

Block S110 recites: receiving a consumable sample, which functions to receive a sample that the user intends to analyze for presence of a harmful substance. In Block S110, the consumable sample is preferably received at an embodiment, variation, or example of the system described in U.S. application Ser. No. 14/498,298, filed on 26 Sep. 2014 and entitled “System and Method for Detection of Target Substance”; however, variations of Block S110 can alternatively include reception of the consumable sample at any other suitable system or process chamber. In Block S110, the consumable sample is preferably in a substantially degraded state (e.g., ground up, liquefied) in order to facilitate extraction of one or more allergen constituents of the consumable sample in a uniform manner that is representative of the allergen constituent(s) of the consumable sample in bulk; however, the consumable sample can alternatively be in any other suitable state of degradation. Furthermore, the consumable sample can be a “fresh” consumable sample (e.g., recently acquired and in a state of low degradation); however, the consumable sample can alternatively be characterized by any suitable state of degradation that still allows for downstream detection of the allergen constituent(s) of the consumable sample. The consumable sample can possess any suitable temperature (e.g., hot, boiling, sizzling, warm, cold, frozen, chilled, room temperature), form (e.g., solid, liquid, gas, emulsion, slurry), consistency (e.g., chewy, creamy, buttery, tough, soft, light, heavy, etc.), texture (e.g., crunchy, crispy, rough, smooth, etc.) and/or any suitable characteristic of consumable items (e.g., food, beverage, cosmetic, etc.).

In relation to Block S110, the consumable sample is preferably a food sample (e.g., a pure food sample, a processed food sample, etc.) potentially containing a harmful substance (e.g., an allergen), and is preferably an unprocessed food sample prior to applying any degradation processes, such that a user (e.g., a food consumer, a food preparer, a manufacturer, a retailer, etc.) can gather an insubstantial volume of a food substance that he/she intends to consume for a meal, and deliver it to a sample processing system for processing and analysis. In this example, the food sample can comprise a mixture of different food items (e.g., different components of an entrée), can comprise a single food item (e.g., a single component of an entrée), and/or can comprise a sequence of different food items (e.g., a sequence of components from an entrée). The food sample can be cored, spooned, cut, tweezed, and/or processed from a bulk volume of food in any other suitable manner. However, in variations, the consumable sample can include any one or more of a: beverage sample (e.g., volume of a mixed drink), cosmetic substance (e.g., volume of makeup, volume of lotion, volume of fragrance, volume of soap, etc.), and any other sample potentially containing a substance that is harmful to the user. In specific examples, the consumable sample can have a volume of between 1 and 7 mL prior to processing (e.g., homogenization, grinding) and treatment with an extraction buffer; however, the consumable sample can alternatively have any other suitable volume. In a specific example, receiving the consumable sample can take 5-30 seconds (e.g., 10 seconds, under 20 seconds, etc.). However, Block S110 can take a shorter amount of time, a longer amount of time, and/or any suitable amount of time.

In a first variation, Block S110 can comprise receiving the consumable sample at a first chamber of a capsule. The capsule can be a component of the indicator module described in Section 3.3 below, or can alternatively be separate and/or coupled to an indicator module. The first chamber is preferably accessible by a user, such that a user can place a consumable sample into the first chamber. The first chamber and/or the capsule can have any suitable shape, dimensions, material, and/or characteristic suitable for receiving a consumable sample. The first chamber is preferably sealable (e.g., with a cap, adhesive, pressure seal, etc.), but can alternatively be exposed and/or have other suitable enclosure characteristics. The first chamber and/or the capsule can have any characteristics of an embodiment, variation, and/or example of the system described in U.S. application Ser. No. 14/498,298, filed on 26 Sep. 2014 and entitled “System and Method for Detection of Target Substance,” or described in U.S. application Ser. No. 14/227,543 filed on 27 Mar. 2014 and entitled “Portable Device for Detection of Harmful Substances”. However, the consumable sample can be received in any suitable manner at a first chamber of a capsule, where the first chamber and the capsule possess any suitable characteristics.

In a second variation, Block S110 can comprise receiving a first consumable sample and receiving a second consumable sample. The first and the second consumable samples can be received at the same or different chambers of a capsule. Further, the first and the second consumable samples can be received simultaneously, serially, or in any suitable order. The first and the second consumable samples are preferably subsequently assessed for different target biomolecules, but can alternatively be analyzed for the same target biomolecule (e.g., performing two analyses on two different samples for the same target biomolecule to increase statistical accuracy). For example, a diner at a restaurant can place a first food type of a meal (e.g., a first consumable sample) into a first chamber of the capsule, and the diner can place a second food type of the meal (e.g., a second consumable sample) into the second chamber of the capsule. The first consumable sample can be analyzed for a gluten-derived analyte, and the second consumable sample can be analyzed for peanut-derived analyte. However, the first and the second consumable samples can be analyzed for any number, type, and/or combination of allergen constituents and/or target biomolecules. After receiving the first and the second consumable samples, the samples can be processed in serial, in parallel, separately, together, or in any suitable relationship. However, receiving the first and the second consumable samples can be performed in any suitable manner.

### 3.2 Extracting a Test Sample.

Block S120 recites: extracting a test sample including an allergen constituent from the consumable sample with an extraction buffer, which functions to extract at least one allergen constituent of the consumable sample received in Block S110, and to prepare the extracted allergen constituent(s) for downstream processing at the allergen indicator module. Regarding Block S120, extraction preferably includes transitioning from an allergen present in a food phase into a solubilized phase in the extraction medium. In Block S120, extraction is preferably performed in an automated process, such that a volume of a test sample generated upon treatment of the consumable sample with the extraction buffer can be directly transmitted to the allergen indicator module for processing (i.e., without any further dilution, without processing with a subsequent reagent prior to detection at the allergen indicator module). Preferably, the extraction buffer is configured to extract a gluten-derived analyte (e.g., Gliadins, hordein, secalin, etc.) efficiently from the consumable sample, and preferably has fluid characteristics (e.g., hydrophilicity, hydrophobicity, viscosity, ionic strength, pH, etc.) that facilitate downstream processing at the allergen indicator module (e.g., at a test strip of the allergen indicator module, using another suitable assay method) in an efficient manner. Alternatively, extraction of a test sample with an extraction buffer can be omitted from the method 100. However, the extraction buffer can additionally or alternatively be configured to facilitate extraction of any other suitable analyte from the consumable sample. Extracting the test sample from the consumable sample with an extraction buffer can be performed at any suitable efficiency level. For example, for a specific amount of allergen in a consumable sample (e.g., per molar), any suitable amount of the allergen (e.g., in terms of percentage of the original allergen amount, etc.) can be extracted into the extraction buffer.

In relation to Block S120, extracting a test sample is preferably performed after receiving the consumable sample. There are preferably no dilution or chemical processing steps between receiving the consumable sample and extracting the test sample with extraction buffer, such that Block S120 can include extracting the test sample upon receiving a consumable sample (e.g., in a homogenized state), and, in response to extracting the test sample, directly transmitting the test sample to an indicator module for processing. Between receiving the consumable sample and before extracting the test sample, Block S120 can include receiving grinded (e.g., by a user) portions of the consumable sample. Additionally or alternatively, Block S120 can include intermediate chemical processing or dilution steps after receiving the consumable sample and before extracting the test sample. However, extracting the test sample can be performed at any suitable time and/or in any suitable relation to other portions of the method 100. In a specific example, extracting the test sample can take 10-30 seconds (e.g., 20 seconds, under 30 seconds, etc.). However, Block S120 can take any suitable amount of time.

In a first variation, Block S120 can include extracting a test sample at an extraction chamber of the capsule. The extraction chamber can be coupled (e.g., physically connected, adjacent, connected by a port, connected by a valve, etc.) to a first chamber in which the consumable sample is received. For example, upon receiving the consumable sample at the first chamber, the consumable sample can be transmitted to an extraction chamber where extraction buffer is received and combined with the consumable sample to extract a test sample. Combination of the extraction buffer with the consumable sample can be aided by a user (e.g., directing the user to shake the module containing the consumable sample and the extraction buffer, instructing the user to press a button releasing extraction buffer, and/or any suitable user action), but can otherwise be independent from user action. The consumable sample can be received at an input port of the extraction chamber, where the consumable sample can be received from a user (e.g., a user transmits the consumable sample to the extraction chamber). Alternatively, the sample can be received from another component of the capsule. Transmission can occur through mechanical actuators, gravity, magnetism, electricity-powered mechanisms, pressure, flow (e.g., porous flow), and/or any suitable mode of transmission. However, extracting the test sample can be performed at a first chamber (e.g., a chamber where the consumable sample is received) of the capsule, at a chamber of the indicator module, and/or at any suitable location through any suitable fashion.

In a second variation, Block S120 can include detecting the consumable sample (e.g., presence of the consumable sample, an amount of the consumable sample) within the extraction chamber. Detecting the consumable sample within the extraction chamber can be in response to receiving the consumable sample at the extraction chamber, but can be performed at any other suitable time. Detecting the consumable sample can include detecting the presence and/or a measureable quantity of consumable sample at the extraction chamber. Detecting the consumable sample can be performed through use of one or more of: pressure sensors, light sensors, optical sensors, touch sensors, magnetic sensors, capacitance, and/or any suitable means of detection. Block S120 can thus include, in response to detecting the consumable sample at the extraction chamber, combining the consumable sample with extraction buffer. For example, upon detection of the presence of consumable sample at the extraction chamber, extraction buffer can be transmitted to the extraction chamber for combination with the consumable sample. However, detecting the consumable sample and/or transmitting the extraction buffer can be performed in any suitable fashion.

In a third variation, Block S120 can include combining the extraction buffer with the consumable sample at the chamber receiving the consumable sample. In an example of the third variation, a first chamber of a capsule receives the consumable sample, and subsequently, extraction buffer is combined with the consumable sample at the first chamber. Alternatively, the extraction buffer can be combined with the consumable sample at any suitable region receiving the consumable sample. Combination of the extraction buffer with the consumable sample at the chamber and/or receiving the consumable sample can be user-aided (e.g., a user performs an action helping the extraction of the test sample through combination of the buffer and the sample) or without the aid of a user. In a specific example of this variation, the first chamber is sealed by a twistable cap, the underside of which includes mechanical protrusions for grinding the consumable sample as the user twists the cap to seal the first chamber. In this specific example, the user can place a consumable sample into a first chamber of a capsule, place the twistable cap on to the first chamber, extraction buffer can be transmitted to the first chamber, and the user twisting the cap to seal the chamber will grind the consumable sample along with mixing the extraction buffer with the consumable sample. Additionally or alternatively, combining the extraction buffer with the consumable sample can include: magnetic stirring, electrical grinding, pulverizing, centrifugation, rocking, agitation, vortexing, combination with pulsing, mixing and grinding, mixing and homogenization, etc. However, combining the extraction buffer with the consumable sample at the region receiving the consumable sample can be performed in any other suitable manner.

Regarding Block S120, in variations of the extraction buffer for gluten characterization in the consumable sample, the extraction buffer can include one or more of: polysorbate (e.g., polysorbate 20, polysorbate 40, polysorbate 60, polysorbate 80, etc.), sodium dodecyl sulfate (SDS), sodium chloride (NaCl), and Tris-Hydrochloride (Tris-HCl), bovine serum albumin (BSA), phosphate buffered saline (PBS), Tween-20, and chaotropic salts (e.g., lithium perchlorate, other perchlorates, guanidine HCL, guanidine thiocyanate, other thiocyandates, urea, etc.). Furthermore, the pH of the extraction buffer is preferably basic, but can alternatively have any other suitable pH (e.g., neutral, acidic). In variations, the concentration of polysorbate (e.g., polysorbate 20) can range from 2-4% in solution, the concentration of SDS can range from 0-4% in solution, the concentration of Tris-HCl can range from 25 mM-200 mM in solution, the concentration of NaCl can range from 0-8M in solution, the concentration of N-lauroylsarcosine can range from 0-4% in solution, the concentration of BSA in solution can range from 0 to 100 mg/ml, and the pH of the extraction buffer can be from 4-11.

In a specific example, the extraction buffer can include one or more of: polyethylene glycol sorbitan monolaurate (e.g., at a concentration of 0-5%), sodium dodecyl sulfate (e.g., at a concentration of 0-2%), sodium thiocyanate (e.g., at concentration of 0-10%), tris(hydroxymethyl)amino-methane (e.g., at a concentration of 0-200 mM), hydrochloric acid (e.g., adjusting the solution to pH 8-12), sodium hydroxide (e.g., adjusting the solution to pH 8-12), and PES bottle filter. However, the extraction buffer can include any suitable component at any suitable concentration in any suitable solution pH.

Furthermore, in relation to Block S120, the extraction buffer can omit ethanol, which can adversely affect antibodies used for detection of the allergen constituent (i.e., for a one-step extraction process without dilution). However, the extraction buffer used in Block S120 can alternatively include ethanol (e.g., 60% ethanol), a compound with a hydroxyl functional group (e.g., mercaptoethanol), and/or any other suitable buffer component that facilitates extraction of the allergen constituent(s) from the consumable sample. For instance, in variations of Block S120, the extraction buffer can include an extraction solution comprising one or more of: guanidine hydrochloride, tris(2-carboxyethyl)phosphine (TCEP), lauroylsarcosine, fish gelatin, polyvinylpyrrolidone (PVP), and any other suitable component. A first specific example of an alternative extraction buffer can include 60% ethanol, a second specific example of an alternative extraction buffer can include 250 mM of 2-mercaptoethanol and 2M guanidine hydrochloride, a third specific example of an alternative extraction buffer can include 20 mM TCEP and 2 M guanidine hydrochloride in saline, a fourth specific example of an alternative extraction buffer can include 5 mM TCEP and 2% N-lauroylsarcosine in saline (pH=7), a fifth specific example of an alternative extraction buffer can include 50 mM TCEP, 2% polysorbate 20, and 0.5% SDS in 50 mM Tris at pH 8.4, and a sixth specific example of an alternative extraction buffer can include 50 mM TCEP, 2% polysorbate 20, and 0.5% SDS in 50 mM Tris at pH 10.

As noted above for Block S120, in alternative variations, the extraction buffer can additionally or alternatively be configured to extract any other suitable allergen or target substance from the consumable sample. For instance, for a peanut-derived allergen (e.g., Ara H1, Ara H2, Ara H3, etc.), the extraction buffer can include one or more: of PBS with NaCl (e.g., 1M NaCl), Tris (e.g., 20 mM Tris), milk (e.g., nonfat dry milk), Tricine, and any other suitable buffer component configured to facilitate extraction. Additionally or alternatively, in variations for other allergens, the extraction solution can comprise any molarity and/or pH of ethanol, BSA (e.g., 0.33 mg/ml BSA, pH 8.6), PBS (e.g., 1×PBS and 1% T20, 1×PBS and 2% T20), phosphate (e.g., 50 mM phosphate, pH 7), borate buffered saline (BBS), and Tris buffer for a dairy-derived allergen (e.g., lactose, casein), a parvalbumin extraction solution for a fish-derived allergen, an ara-h2 extraction solution for a nut derived allergen, an egg protein extraction solution for an egg-derived allergen (e.g., ovomucoid protein, ovalbumin protein, ovotransferrin protein, lysozyme protein), a tropomyosin extraction solution for a shellfish-derived allergen, and/or any other suitable extraction solution for any other allergen. Furthermore, variations of Block S120 can incorporate reagent(s) configured for any one or more of: lysing of a sample, solubilization of a sample, buffering of a sample, dilution of a sample, and any other suitable reagent(s).

In Block S120, extracting with the extraction buffer preferably includes combining the extraction buffer with the consumable sample (e.g., by magnetic bar mixing, by electrical grinding, by centrifugation, by rocking, by agitation, by vortexing, combination with pulsing, mixing and grinding, mixing and homogenization, etc.) to extract a protein contributing to an allergic response (e.g., gliadin) from the consumable sample, wherein the extraction process occurs within a period of 5 minutes (e.g., 1-2 minutes) and an output of extraction can be directly applied to the allergen indicator module without any subsequent reagent treatment, dilution, or other processing. However, alternative variations of Block S120 can include any other suitable number of extraction and processing steps to generate the test sample comprising the allergen constituent prior to substance detection in Block S140.

### 3.3 Transmitting the Test Sample.

Block S130 recites: transmitting the test sample to an allergen indicator module S130, which functions to deliver the test sample to a module that can provide a detectable signal pertaining to characteristics of the allergen constituent of the consumable sample. The allergen indicator module implemented in Block S130 can include a lateral flow system including a test strip; however, the allergen indicator module implemented in Block S130 can alternatively comprise any one or more of: a chromatography device, a plate assay device (e.g., including an immunosorbent assay plate), and any other suitable immunoassay device configured to generate a detectable signal in response to antibody-antigen binding. Devices (e.g., an immunoassay device) of the allergen indicator module can be coupled to and/or in communication with the extraction chamber, a chamber receiving the consumable sample, and/or any suitable portion of the capsule and/or other component. In variations, transmitting the test sample to the allergen indicator module can include passing the test sample through a port (e.g., a valved port) in facilitating contact between the sample and a detection region of the allergen indicator module (e.g., a detection region of a test strip). In other variations, transmitting the test sample can additionally or alternatively include transmission by mechanical actuators, gravity, magnetism, electricity-powered mechanisms, pressure, capillary action, flow (e.g., porous flow), user-aided transmission (e.g., flipping, shaking, twisting, etc.) and/or any suitable mode of transmission. In further variations, the immunoassay device is a test substrate embedded within the capsule, wherein directly transmitting the sample comprises directly transmitting the sample, through a valve, to a sample pad of the test substrate. Some embodiments, variations, and examples of test sample transmission to an allergen indicator module are described in U.S. application Ser. No. 14/498,298, filed on 26 Sep. 2014 and entitled “System and Method for Detection of Target Substance”. In a specific example, transmitting the test sample to an allergen indicator module can take 0.5-5 seconds (e.g., 1 second, under 5 seconds, etc.). However, Block S130 can take any suitable amount of time.

In one variation, Block S130 can include directly transmitting the test sample to a test substrate of the indicator module (e.g., in a lateral flow format). The test sample is preferably directly transmitted from an extraction chamber of a capsule to the sample pad integrated with the capsule and in communication with the extraction chamber of the indicator module, but can additionally or alternatively be transmitted to and/or from any suitable region of the any suitable component. In this variation, the sample pad is preferably integrated with the capsule in a manner such that a user need not interact with the sample pad to obtain information regarding the presence and/or quantity of an allergen constituent in a consumable sample. Directly transmitting is preferably performed upon extraction of the test sample, but can otherwise be performed at any suitable time.

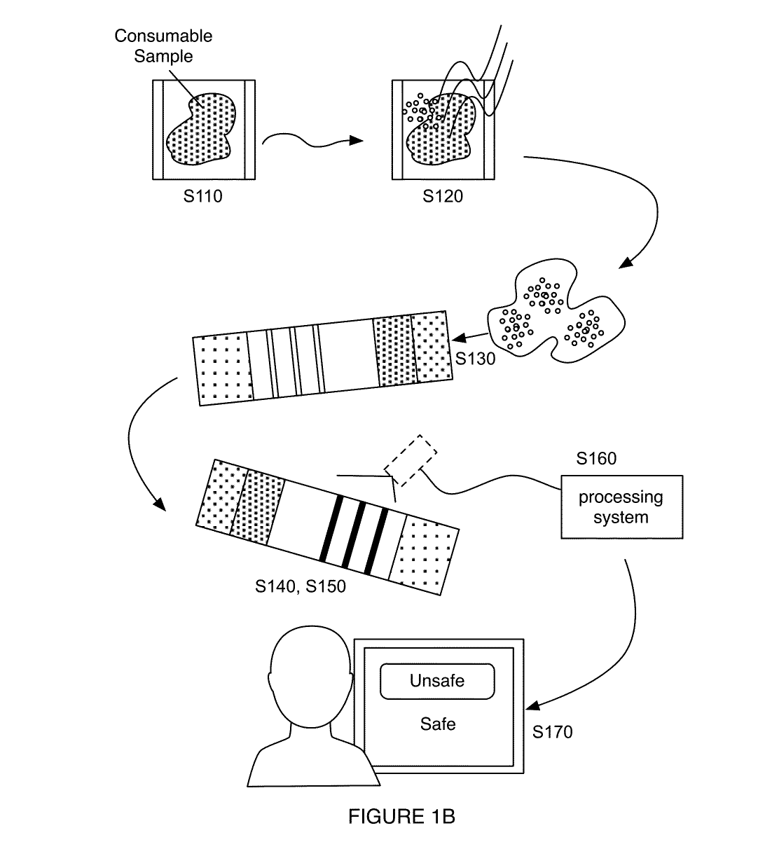

In one variation of the allergen indicator module used in Block S130, as shown in FIG. 2, the allergen indicator module comprises a lateral flow element comprising: a membrane including a detection region, an absorbent region coupled to a downstream end of the membrane (in order to facilitate flow through the membrane), a sample pad disposed at an upstream end of the membrane and configured to receive and transmit the test sample through a conjugate pad and into the membrane, and a back card that serves as a substrate for the membrane, wherein the back card can couple to a cover that functions to maintain positions of elements of the lateral flow element. In extensions of this variation, the lateral flow element can include desiccant elements and/or heat-protective elements that prevent moisture and/or heat damage to the lateral flow element. One or more desiccant elements can be included adjacent the lateral flow element to enable moisture protection. However, any number of desiccant elements can be used at any suitable position to prevent moisture and/or heat damage to any suitable component.

Regarding Block S130, in variations of the lateral flow element, the membrane can be composed of a permeable material (e.g., nitrocellulose membrane, CN95 membrane, CN140 membrane, etc.), the absorbent region can be composed of a permeable material (e.g., Millipore C095, EMI 30250, etc.), and the sample and conjugate pads can be composed of a permeable material (e.g., fibrous material, porous material, etc.). In specific examples, the sample and conjugate pads can be integrated and can comprise a commercially available sample pad material (e.g., Ahlstrom 6615, Ahlstrom 6613, Ahlstrom 8950, Ahlstrom 8951, Millipore G041). However, one or more of: the membrane, absorbent region, sample pad, and conjugate pad can be custom-made and/or comprise any suitable material of any suitable permeability.

In relation to Block S130, the dimensions of the lateral flow element (e.g., of the membrane, of the absorbent region, of the sample pad, of the conjugate pad, etc.) are preferably configured to provide a suitable level of fluid wicking behavior, in order to rapidly generate detectable signals in an accurate manner. Preferably, the width of the lateral flow element (e.g., of the membrane) is configured to provide suitable behavior in fluid wicking through the lateral flow element. In a specific example, the lateral flow element has a length of 35 mm and a width of 5 mm, wherein the membrane of the lateral flow element has a length of 25 mm and a width of 5 mm, the absorbent pad has a length of 9 mm with a 2 mm overlap with a downstream portion of the membrane, the conjugate pad has a length of 8 mm and has a 3 mm overlap with an upstream portion of the membrane, and the sample pad is coupled to an upstream end of the conjugate pad and has a 5 mm overhang from the back card, which has a length of 35 mm. In the specific example, the 25 mm membrane was applied to a 35 mm back card so that a first edge of membrane was displaced 16 mm from the bottom edge of the back card and a second edge of membrane was displaced 20 mm from top edge of the back card. An 8 mm Millipore C095 wicking pad, as the absorbent region, was then placed overlapping the second edge of the membrane, and an 8 mm conjugate pad was placed overlapping the first edge of the membrane by 3 mm. Finally, excess material of the back card was cut off. In extensions of the specific example, A 26 mm wide clear covertape was placed across the membrane and overlapping onto both the conjugate and wicking pad by 3 mm, in order to facilitate maintenance of the positions of elements of the lateral flow element. However, in variations of the specific example, portions of the lateral flow element can have any other suitable dimensions and overlap with each other in any other suitable manner, and/or can be assembled in any other suitable manner.

Regarding Block S130, in more detail, the conjugate pad functions to provide bioactive substrates (i.e., conjugate particles with antibodies) that form complexes with the allergen constituent of the test sample, and flow toward the detection region of the membrane for subsequent binding to an antibody paired with the antibodies of the complexes. In one variation, the substrates comprise cellulose nanobeads in enabling cellulose nanobead conjugation (CNB); however, in other variations, the substrates can comprise any other suitable material (e.g., gold particles, fluorescence or absorption based latex particles such as europium-latex particles, etc.). In one example, the conjugate pad was prepared as follows: a conjugate buffer (e.g., a buffer including 200 mM Borax at pH 9, a buffer including polysorbate 20 and BSA, etc.) was applied to the conjugate pad at room temperature, dried at 40° C. for one hour, and a conjugate solution comprising the desired concentration of CNBs conjugated with a suitable antibody was prepared and combined with sucrose powder (e.g., 0.2 g sucrose/2 mL of conjugate solution) and trehalose to facilitate rehydration and stability of the conjugate, respectively. The prepared conjugate solution was applied by airjet onto the buffered and dried conjugate pad using a 10 micrometer spigot, a pressure of 2 psi, a volume flow of 10 μL/cm, and a distance of 56 mm from the conjugate pad. The conjugate pad was then dried at 40° C. and coupled with other portions of the lateral flow element, as described above. In another example, the prepared conjugate solution was applied by printing onto the conjugate pad directly at 3.5 ul/cm. Additionally or alternatively, in some variations, the conjugate particles of the conjugate pad can comprise Europium incorporated carboxylated-latex particles that function to enable detection of a target analyte of the consumable sample Conjugate particles of the conjugate pad can possess any suitable optical density (e.g., OD 15, OD 25, etc.). However, the conjugate pad can be prepared in any suitable fashion and can comprise any suitable bioactive substrate possessing any suitable characteristic.

With respect to Block S130, in specific examples including detection of peanut-derived allergen constituents (e.g., Arachis Hypogaea 1-8), the conjugate pad can comprise europium-latex particles conjugated with antibodies (e.g., 2F7 monoclonal antibody (InBio)) configured to pair with peanut-derived allergens, where the antibody solutions can possess antibody concentration of 1 mg/ml diluted in 1×PBS and 1% T-20. In other specific examples including detection of peanut derived allergen constituents, the conjugate pad can comprise colloidal gold particles, where gold conjugates possess OD15-26. In specific examples including detection of dairy-derived allergen constituents (e.g., casein protein), the conjugate pad can comprise colloidal gold particles (e.g., where gold conjugates possess OD1 to OD30) conjugated with antibodies (e.g., Polyclonal Anti-Casein bs-0813R (Bioss)) configured to pair with dairy-derived allergen constituents. In other examples for detection of dairy-derived allergen constituents, the conjugate pad can comprise europium-latex particles conjugated with polyclonal anti-casein antibodies. However, conjugate pads containing bioactive substrates configured to react with peanut-derived allergen constituents and/or dairy-derived allergen constituents can include any suitable bioactive substrate, antibody, and/or buffer with any suitable characteristic. However, Block S130 can include preparing the conjugate pad with any other suitable particles appropriate for any other suitable target component of the sample.

In more detail, the detection region of the membrane functions to provide regions for binding and signal generation that is indicative of characteristics of the allergen constituent of the test sample. The detection region preferably comprises a control region configured to capture any particle flowing into the control region and indicate proper operation of strip, and a test region comprising bound antibodies paired with the antibodies of the complexes from the conjugate pad, that functions to capture complexes specific to the allergen constituent of the test sample. Additionally, the detection region can comprise a competitive region comprising a pre-applied amount of target analyte associated with the allergen constituent of the test sample, wherein the competitive region can enable detection of a false-negative result due to a hook effect created when the test sample has an extremely high concentration of the allergen constituent. Furthermore, the detection region can also facilitate detection of one or more fragmented antigens and/or other fragmented molecules of interest, in some variations. In particular, the competitive region can be used when a sandwich assay cannot be formed (e.g., with hydrolyzed gluten that lacks enough epitopes per protein to form a sandwich assay, the competitive region can still function properly because it requires only one epitope). In a specific example, related to gluten detection in the test sample, the competitive region can comprise printed gliadin (or another associated protein with a lower affinity for the antibody, or another protein associated with another allergen), which can be used to detect false-negatives in relation to the test region. In particular, the affinity of printed gliadin in the competitive region should be less than or equal to that of the affinity of gliadin in the test sample, in order to avoid competitive binding that could produce a false signal. However, any suitable biomolecule with any suitable affinity (e.g., a biomolecule with a higher affinity for the antibody, with lower affinity, with the same affinity, etc.) can be used in the competitive region.

In another specific example, related to peanut detection in the test sample, the competitive region can comprise printed Ara H2 (or Ara H1, Ara H3, or associated protein with a lower affinity for the antibody, or another protein associated with another allergen), which can be used to detect false-negatives in relation to the test region. In particular, the affinity of printed Ara H2 in the competitive region can be less than or equal to that of the affinity of peanut-derived allergen (e.g., Ara H2, Ara H2, Ara H3) in the test sample, in order to avoid competitive binding that could produce a false signal. However, any suitable affinity can be used. Use of the control region, the test region, and the competitive region in generating signals indicative of characteristics of the allergen constituent in the test sample are described further in relation to Block S140 below.

In the detection region of the variation of the allergen indicator module described above, the control region, the test region, and/or the competitive region are preferably well-defined regions of the membrane, and positioned so as to not generate illegible detection signals. In a first variation, the control region, the test region, and the competitive region comprise lines oriented perpendicular to a flow direction through the membrane, wherein the line associated with each region is displaced from the other lines to prevent signal interference (e.g., due to protein smearing across lines). In specific examples, the line associated with each of the control region, the test region, and the competitive region was applied at a volume flow rate of 1 μL/cm (e.g., using a BioDot dispenser), and each line is spaced at least 4 mm from an adjacent line to prevent signal interference. In a specific example, as shown in FIG. 2, the line for the control region is displaced ˜7 mm from a downstream end of the membrane, the line for the competitive region (i.e., hook line region) is displaced ˜4 mm upstream of the control region, and the line for the test region is displaced ˜4 mm upstream of the competitive region.

In a second variation of Block S130, the control region, the test region, and the competitive region comprise dots positioned on the membrane, wherein the dot associated with each region is displaced from the other dots to prevent signal interference (e.g., due to protein smearing across lines). In specific examples, the dot associated with each of the control region, the test region, and the competitive region was applied onto the membrane by diluting antibody solutions to 0.5 mg/ml in 10 mM phosphate buffer, and applying 1 ul to a mid-region (e.g., away from the ends) of the membrane using a micropipette. Alternative variations of the control region, the test region, and/or the competitive region can, however, have any other suitable morphology (e.g., polygonal morphology produced by printing, character-shaped morphology produced by printing, etc.). In other specific examples concerning capture of peanut-derived allergens using corresponding antibodies, dots associated with any of the control region, the test region, and the competitive region can be applied onto the membrane by diluting antibody solutions to 0.5 mg/ml-2 mg/ml, and applying 1 ul per spot or 1 ul/cm to appropriate regions using a micropipette. However, any suitable concentrations of antibody, and any suitable volumes of dot application can be used in preparing regions of a lateral flow device.

Regarding Block S130, the control region is preferably configured downstream of the test region and/or the competitive region, so as to not interfere with capture of a representative portion of the allergen constituent-conjugate complexes at the test region and/or competitive region; however, the control region can alternatively be positioned relative to the test region and/or competitive region in any other suitable manner. Furthermore, the detection region can have any suitable number of control regions, test regions, and/or competitive regions, associated with the same or multiple target analytes (e.g., of different allergens) and configured in any other suitable manner. Additionally, each of the control region, the test region, and the competitive region can develop in any suitable order, in relation to other regions of the detection region. Different conjugate particles emitting at different colors (e.g., at different wavelengths) can be used for detection of different target analytes (e.g., analytes corresponding to different allergens, different analytes corresponding to the same allergen, etc.). For example, latex particles with lanthanide chelate dyes incorporated (e.g., Eu, Sm, Tb, and Dy dyed particles) can be used. Additionally or alternatively, other nanoparticle labels with other lanthanide-specific fluorescence properties, or other particles with other fluorescence properties, can be used. In a specific example, latex particles with incorporated lanthanide chelate dyes are used with any suitable number of control regions, test regions, and/or competitive regions. In this specific example, a single source of light (e.g., at 360 nm) can excite the latex particles with incorporated lanthanide chelate dyes, causing the dyes to emit at different wavelengths. An optical filter can distinguish the emission lights and capture the different colors simultaneously. However, any suitable number of biomolecules with any suitable emission characteristic can be used in analyzing any suitable number and/or type of analyte.

With respect to Block S130, in relation to the detection region and the conjugate pad, the antibody pair preferably comprises custom antibodies configured for detection of gluten-derived analytes with appropriate kinetic behavior. However, the antibody pair of the conjugate pad and the detection region can additionally or alternatively comprise a commercially available antibody, such as XGY1 monoclonal antibody (Zedira GmbH) at 1 mg/mL and goat anti-mouse immunoglobulin (IgG) at 0.5 mg/mL at the test region of the detection region, and 4F3 monoclonal antibody (Thermo Scientific, CN HYB314-02-02) conjugated to CNBs at the conjugate pad. Alternatively, in other variations for detection of a gluten-derived allergen constituent in the test sample (e.g., anti-Gliadins), antibodies used in antibody pairs at the conjugate pad and the test region of the detection region can include one or more of: 14D5 monoclonal antibody (Thermo Scientific, CN HYB314-01-02), Rabbit anti-Gliadin polyclonal antibody (Biossusa, Inc, CN BS-13374R, LN 131121), Rabbit anti-Gliadin polyclonal antibody (Sigma Aldrich, CN G9144, LN 122M4828), goat anti-rabbit IgG (H+L) (Lampire Biologicals, CN 7455607, LN 13C51015), R5 monoclonal antibody (e.g., for detection of wheat, rye, and barley), G12 antibody (e.g., for detection of wheat rye, barley, and some oats), a monoclonal 43G12 antibody, and any other suitable complementary antibody for Gliadins. In a specific example, a custom antibody pair can comprise a monoclonal 43G12 antibody paired with a 43G12 monoclonal antibody (i.e., as a self-pair). In another specific example, a custom antibody pair can comprise a polyclonal antibody (e.g., a goat polyclonal antibody) paired with a 43G12 monoclonal antibody. In another specific example, the custom pairing can comprise 14G11 on the conjugate and 13F on the detection region. However, the antibodies can include IgG1, IgG2b, other isoforms, or any other suitable antibody.

Regarding Block S130, other antibody pairs can, however, be used in variations of the method 100 involving detection of a gluten-derived target and/or any other suitable allergen-derived target in the test sample. Furthermore, antibody pairs used in the method 100 can comprise monoclonal antibodies and/or polyclonal antibodies (e.g., mono-mono-pairs, poly-poly pairs, mono-poly pairs). Additionally, a recognition element can comprise one or more of an aptamer, a peptide, and any other suitable component. For instance, for a peanut-derived allergen (e.g., Arachis Hypogaea 1-8), antibody pairs in variations of the method 100 can include one or more of: 2C12 monoclonal antibody (InBio), 2F7 monoclonal antibody (InBio), chicken immunoglobulin Y polyclonal antibody (LifeSpan BioScience), chicken immunoglobulin Y polyclonal antibody (antibodies online), rabbit immunoglobulin G polyclonal antibody (Abcam), hen immunoglobulin Y polyclonal antibody (Agrisera), rabbit polyclonal antibody (Sigma Aldrich), rabbit immunoglobulin G polyclonal antibody (MyBioSource), goat immunoglobulin G polyclonal antibody (MyBioSource), mouse immunoglobulin G monoclonal antibody (InBio), and any other suitable antibody for binding of a peanut-derived allergen analyte.

In variations for detection of a dairy-derived allergen constituent (e.g., casein) in the test sample, antibodies used in antibody pairs at the conjugate pad and the test region of the detection region can include one or more of: Rabbit Polyclonal Anti-Casein bs-0813R (Bioss), Rabbit Polyclonal Anti-alpha s1 Casein bs-10033R (Bioss), Rabbit Polyclonal Anti-kappa Casein GTX-60145 (GeneTex), Rabbit Polyclonal Anti-Casein ab-166596 (Abcam), BF-A anti-casein monoclonal antibody (Biofront), BF-B anti-casein monoclonal antibody (Biofront), generic anti-casein antibodies, anti-alpha casein antibodies, anti-kappa casein antibodies, and/or any suitable antibody for interaction with dairy-derived allergen constituents.

With respect to Block S130, antibodies used in the detection region (e.g., antibodies plated at the testing region of the detection region) can be of the same or different type of antibody as the antibodies used at the conjugation pad. However, any suitable combination of antibodies at any suitable concentration can be used for portions of the conjugate pad and/or the detection region.

In a third variation, Block S130 can include reacting the test sample with colored particles (e.g., colored moieties, fluorescent moieties, etc.) labeled with the allergen constituent and/or a suitable analogue. Subsequently, when the test sample flows through a test region plated with antibodies paired with the allergen constituent and/or the analogue, unlabeled allergen constituent in the test sample can prevent further binding on the antibody sites. Colored particles will thus be unable to bind to the antibodies, and the testing region would generate an activated indication characteristic in negative samples. However, the test sample can react with any suitable sets of biomolecules to determine the presence and/or quantity of constituents in the test sample.

In a fourth variation, Block S130 can include transmitting the test sample through a substrate comprising microfluidic channels. An extracted test sample can be transmitted through microfluidic channels of patterned-paper, lab-on-a-disc, lab-on-a-chip, and/or any suitable microfluidic devices. In a specific example, a test sample is transmitted to the upstream start of microfluidic channels passing through a testing region, competitive region, and or a control region of a microfluidic device. The microfluidic device is preferably embedded within the allergen indicator module, such that a test sample can be directly transmitted from an extraction chamber to an upstream input region of the microfluidic device. Alternatively, microfluidic-based technology can be implemented with lateral flow principles of the allergen indicator module. However, microfluidic-based technology can be applied in any suitable manner to any suitable component to assess target biomolecules in a consumable sample.

In a fifth variation, Block S130 can include reacting the test sample with a recognition moiety in fluorescence resonance energy transfer. The recognition moiety (e.g., an aptamer) preferably has an affinity for the target analyte (e.g., gluten allergen, a dairy-derived allergen, a nut allergen, a fish allergen, an egg-derived allergen, a soy derived allergen, a peanut-derived allergen, shellfish-derived allergens, etc.). Further, the recognition moiety preferably comprises two or more molecules (e.g., a donor molecule, acceptor molecule, etc.), such that once an antigen binding event occurs, the recognition moiety undergoes a conformation change causing the distance between the two or more molecules to decrease. Excitation of the donor molecule preferably causes the acceptor molecule to emit a specific-colored light (e.g., at a particular wavelength) that can be captured by a reader (e.g., customized reader, standard reader, etc.). Additionally or alternatively, a bead-based proximity assay such as AlphaScreen can be used in detecting the presence and/or amount of target analytes.

However, other variations of Block S130 can additionally or alternatively be implemented in any other suitable manner.

### 3.4 Generating a First Indication Characteristic.

Block S140 recites: generating an indication characteristic of presence of the allergen in the consumable sample upon reacting the allergen constituent with a probe at the allergen indicator module. Block S140 functions to provide a signal (e.g., an optically detectable signal, an electronically detectable signal, a thermally detectable signal, an audibly detectable signal) indicative of characteristics of presence of the allergen in the consumable sample. Preferably, the signal is an optically detectable signal (e.g., a fluorescent signal, a colored signal from a colored particle, etc.), and in variations, can include a signal detectable under visible light or detectable upon irradiation with excitation wavelengths of light (e.g., to provide an emission response). The signal can be detectable using a single excitation source or multiple excitation sources. Additionally or alternatively, the signal can be detected through non-optical means (e.g., an indication that is a physical modification to a testing region, etc.). Detected signals can be direct signals, indirect signals, a combination of direct and indirect signals, and/or any other type of signal. However, the signal can additionally or alternatively be detectable in any other suitable manner. The probe can be any one or more of: an antibody, bioconjugate, enzyme, activity probe, receptor, optical (e.g., fluorescent, colored, etc.) probe, and/or any suitable biomolecule probe. However, any type, combination, and/or number of probes can be used in generating the indication characteristic.

Block S140 can additionally or alternatively include amplifying the indication characteristic to increase detectability of a target molecule. Means of amplification include: enzyme-based amplification (e.g., through use of horseradish peroxidase, etc.), a silver nitrate/nitrite system, macrofluorophore labeling, and/or other suitable means of amplification. However, any suitable signal can be amplified using any suitable mechanism.

In relation to Block S140, the first indication characteristic is preferably generated at a testing region of the detection region of an immunoassay device. Alternatively, indication characteristics (e.g., the first, the second, and the third indication characteristics) can be generated at a competitor region, control region, and/or any suitable region of an immunoassay device. Multiple indication characteristics can be generated at a given region of the immunoassay device. An indication characteristic can be generated at a testing line, dot, well, area, three-dimensional region, and/or any suitable region.

Block S140 can include generating the first indication characteristic before generating any other indication characteristics (e.g., in variations where the sample flows to different regions of a test pad sequentially). Alternatively, the first indication characteristic can be generated simultaneously in time as one or more other indication characteristics. In a first example, a first indication characteristic at a testing region can be generated in parallel with a different indication characteristic at a control region of the immune assay. In a second example, a first indication characteristic at a first testing region can be generated for a first consumable sample simultaneously with an additional indication characteristic at a second testing region for a second consumable sample. In a third example, the first indication characteristic for a first consumable sample can be generated in parallel with the extraction of a second testing sample from a second consumable sample that has been received at the extraction chamber of the capsule (e.g., a capsule receives a first and a second consumable sample to analyze in serial while performing different portions of the method for the consumable samples in parallel). However, the first indication characteristic can be generated at any suitable time and in any suitable relation to generation of other indication characteristics and/or portions of the method 100. In a specific example, the wait time for generating a sufficiently activated indication characteristic can take 5-10 seconds (e.g., 5 seconds, under 10 seconds, etc.). However, Block S140 can take any suitable amount of time.

### 3.5 Generating a Second Indication Characteristic.

Block S150 recites: generating a second indication characteristic upon interfacing the test sample with a bioconjugate configured to react with the allergen constituent at the immunoassay device. The bioconjugate can comprise an antibody, bioconjugate, enzyme, activity probe, receptor, optical (e.g., fluorescent, colored, etc.) probe, paired allergen constituents (e.g., antibodies paired with gliadin), paired biomolecules, and/or any suitable constituent. However, a second indication characteristic can be generated upon interfacing the test sample with any suitable probe and/or biomolecule.

Generating the second indication characteristic is preferably performed at a competitive region of an immunoassay device, where the bioconjugates are plated at the competitive region (e.g., as a line, dot, area, etc.). The second indication characteristic is can be generated after generating a first indication characteristic at a testing region, and before generating a third indication characteristic at a control region of the immunoassay device. In such a configuration, the competitive region can enable accommodation of the hook effect (e.g., an effect causing a false-negative result due to the test sample having a very high concentration of allergen constituent) by identifying the cause of a low signal detected at a testing region of the indicator module. The analysis of the indication characteristics generated at the testing region, competitive region, and/or control region preferably allows a distinction between whether a low signal at a testing region is because of a low amount of allergen constituent in the testing sample or because of an excessively high amount of allergen constituent. However, the second indication characteristic can alternatively be generated before, during, or after any suitable portion of the method 100. In a specific example, the wait time for generating a sufficiently activated second indication characteristic can take 5-10 seconds (e.g., 5 seconds, under 10 seconds, etc.). However, Block S150 can take any suitable amount of time. In another example, the method 100 can include generating a first indication characteristic of presence of the target substance in the consumable sample upon interfacing the test sample with a probe configured to react with the first allergen constituent at a first testing region of the immunoassay device; and proximal in time to generating the first indication characteristic, generating a second indication characteristic upon interfacing the test sample with a bioconjugate configured to react with the first allergen constituent at a first competitive region of the immunoassay device, wherein the bioconjugate comprises an antibody and a biomolecule with a biomolecule affinity for the antibody less than or equal to the first allergen constituent affinity for the antibody. However, any suitable biomolecule affinity can be used.

The second indication characteristic is preferably generated at a detection line along a substrate of a of a lateral flow strip, where the test line and the detection line are at the common substrate. Alternatively, the second indication characteristic can be generated at a separate chamber (e.g., well) of an immunoassay device. However, the second indication characteristic can be generated at any suitable region.

In variations with a lateral flow device, Block S150 can include flowing the test sample from the testing region to the competitive region positioned downstream the testing region of the immunoassay device. In variations with or without a lateral flow device, Block S150 can include transmitting the test sample to the competitive region through one or more transmission mechanisms including: mechanical actuators, gravity, magnetism, electricity-powered mechanisms, pressure, capillary action, flow (e.g., porous flow), user-aided transmission (e.g., flipping, shaking, twisting, etc.) and/or any suitable mode of transmission. However, interfacing the test sample with the bioconjguate can be performed in any suitable manner.

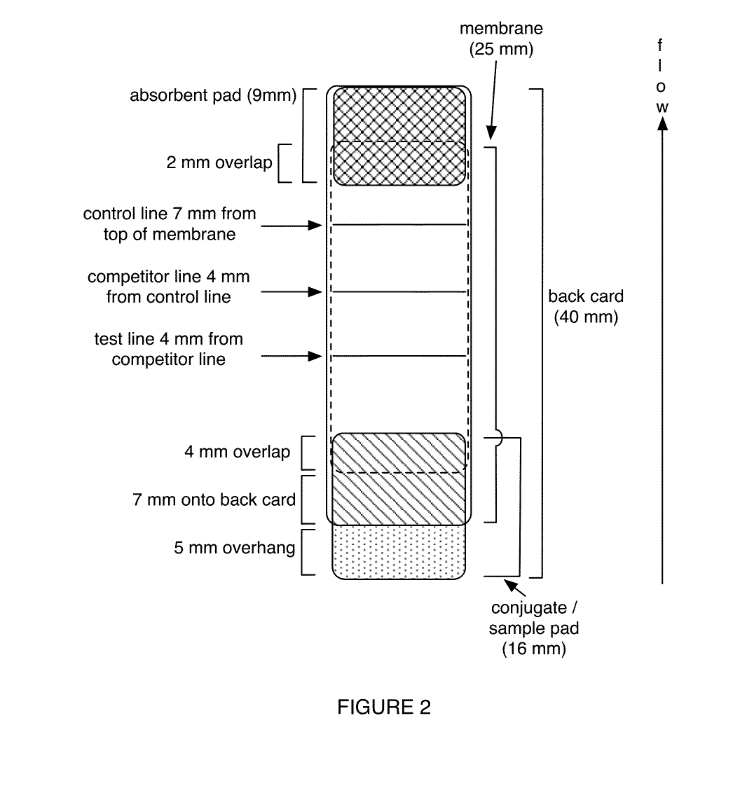

Regarding Block S150, in a variation associated with the lateral flow element described in relation to Block S130 above, the indication can comprise a pattern derived from the configuration of the control region, the test region, and/or the competitive region of the detection region of the membrane. As shown in FIG. 3, activation of the control region and the competitive region, without activation of the test region by binding of antibody-conjugated gliadins in the test sample with antibodies at the test region is detectable as a negative result. Also shown in FIG. 3, activation of the control region, the competitive region, and the test region by binding of antibody-conjugated gliadins in the test sample with antibodies at the test region is detectable as a positive result. Also shown in FIG. 3, activation of the control region and the test region by binding of antibody-conjugated gliadins in the test sample with antibodies at the test region, with saturation of the competitive region (e.g., due to a high concentration of gliadins in the test sample) is detectable as a high positive result. Also shown in FIG. 3, activation of the control region, with saturation of the test region and the competitive region (e.g., due to a very high concentration of gliadins in the test sample) is detectable as a very high positive result. Finally, as shown in FIG. 3, activation of any of the test region and the competitive region, without activation of the control region is detectable as an invalid result, and the test should be re-run. In Block S140 and/or Block S150, the signal of the indication can be temporally monitored in order to determine kinetics of reaction between allergen constituents of the test sample and the test region of the detection region. For instance, temporal aspects of the signal generated can be determined upon measurement of the signal over time.

In another variation, Block S160 can include generating a third indication characteristic of presence of the target substance in the consumable sample upon interfacing the test sample with a second probe configured to react with a second allergen constituent at a second testing region of the immunoassay device; after generating the third indication characteristic, generating a fourth indication characteristic upon interfacing the test sample with a second bioconjugate at a second competitive region of the immunoassay device, wherein generating the analysis of the first and the second indication characteristics comprises generating the analysis of the third and the fourth indication characteristics.

In a further variation, Block S160 can include extracting a first test sample including a first target biomolecule from combining a first portion of a consumable sample with a first extraction buffer, extracting a second test sample including a second target biomolecule from combining a second portion of the consumable sample with a second extraction buffer; and generating a third indication characteristic (e.g., where the first and the second indication characteristics indicate the presence of the first target biomolecule) of presence of the second target biomolecule in the consumable sample upon interfacing the second test sample with a second probe configured to react with the second target biomolecule. However, any number and/or type of indication characteristics can be generated using any number and/or combination of probes, bioconjugates, and biomolecules.

### 3.6 Generating an Analysis of the Indication.

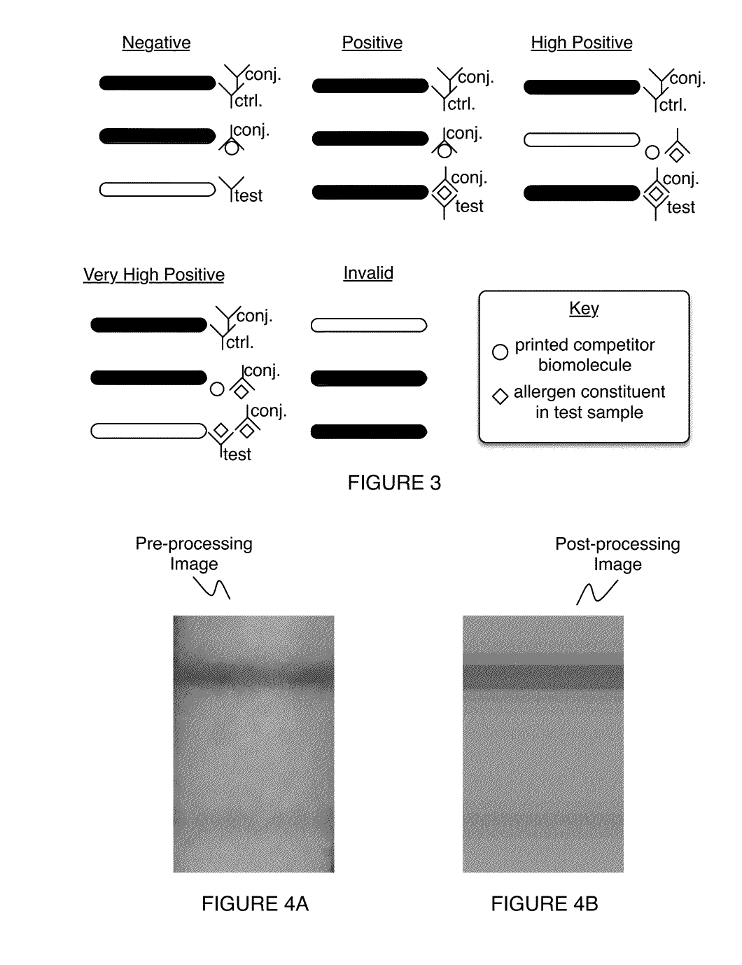

Block S160 recites: generating an analysis of the indication, including at least one of a presence of and an amount of the allergen present in the consumable sample. Block S160 functions to process signals associated with the indication generated in Block S140 and/or Block S150, in order to generate an analysis that provides information regarding presence of one or more harmful substances within the test sample. Block S160 preferably includes generation of an analysis, as in an immunoassay that involves analysis of optically detectable signals produced upon antibody-analyte binding; however, Block S160 can additionally or alternatively incorporate any other suitable analysis. Block S160 is preferably implemented using embodiments, variations, or examples of the system, comprising an optical sensor and a processing and control system described in U.S. application Ser. No. 14/498,298, filed on 26 Sep. 2014 and entitled “System and Method for Detection of Target Substance”; however, Block S160 can alternatively be performed using any other suitable system. As such Block S360 can include any one or more of: denoising, filtering, smoothing, clipping, convoluting, deconvoluting, standardizing, detrending, resampling, and performing any other suitable signal-processing operation on output signals from an optical sensor in communication with the allergen indicator module. In variations of Block S160 involving image data (e.g., image data of the detection region), Block S160 can comprise filtering and/or conditioning image data for sharpness, saturation, edge-finding, intensity, and/or any other suitable image enhancement, examples of which are shown in FIGS. 4A and 4B.

In relation to Block S160, generating an analysis is preferably performed upon the first and the second indication characteristics being generated in Block S140 and Block S150. Additionally or alternatively, generating the analysis can be performed after a first, second, and third indication characteristic have been generated, corresponding to a testing region, a competitive region, and a control region, respectively, of an immunoassay device. Additionally or alternatively, generating the analysis can be performed throughout the user session with the immunoassay device, or dynamacially as the test progresses. However, generating the analysis can be performed at any suitable time, such as, after a predefined time period (e.g., defined by a manufacturer) or automatically determined time period (e.g., after threshold level of saturation at the detection region has been reached) has elapsed after a test sample has begun reacting with a test region and/or competitive region. In a specific example, generating the analysis can take 2-10 seconds (e.g., 5 seconds, under 10 seconds, etc.). However, Block S160 can take any suitable amount of time.

Regarding Block S160, the analysis can be generated at the allergen indicator module (e.g., with an optical sensor and processor of the indicator module), at a separate component, and/or at any suitable device. In a first variation, Block S160 can include generating an analysis of the indication at an analysis device coupled to the allergen indicator module. The allergen indicator module (e.g., capsule with integrated test strip) can preferably be inserted into the analysis device, such that the detection region of the immunoassay device of the allergen indicator module is within a detection field of view of optical sensors of the analysis device. Additionally or alternatively, the analysis device can perform the analysis when the analysis device is physical separated (e.g., without insertion of the allergen indicator module into the analysis device) from the allergen indicator module. In a specific example, the method 100 can comprise receiving a consumable sample at a first chamber of a capsule (e.g., an allergen indicator module), extracting a test sample with an extraction buffer, transmitting the test sample to an immunoassay device embedded in the allergen indicator module, receiving the allergen indicator module at an analysis device (e.g., from a user inserting the allergen indicator module into the analysis device), and generating an analysis of the indication at the analysis device (e.g., through optical sensors and a processor of the analysis device). In another specific example, Block S160 can comprise, within a sample analysis device surrounding the capsule and including an optical sensor configured proximal the sample pad, generating an analysis of the indication. However, the analysis device and allergen indicator module can otherwise be coupled in forming a suitable system for generating the analysis of the indication.

In a second variation, Block S160 can comprise generating an analysis of the indication at a user device of the user. The user is preferably a user consuming the consumable sample, where the consumable sample can be received from the user. The user device can be a smartphone, smartwatch, tablet, desktop, or any other suitable device. The analysis is preferably generated with an optical sensor (e.g., a camera) and a processor of the user device. In an example of the second variation, Block S160 can comprise, receiving an image, captured from a user device, of the testing region of the immunoassay device, and generating an analysis of the indication based on the image. In a specific example, a user can transmit a consumable sample to a chamber of a capsule, the test sample can be analyzed at the immunoassay device of the capsule, and the user can take a picture with their smartphone of the testing region of the immunoassay device (e.g., through an optically transparent wall of the capsule). An application on the user device can generate the analysis of the indication based on the image. However, any suitable device can capture images of the testing region and/or generate an analysis based on the image or images.

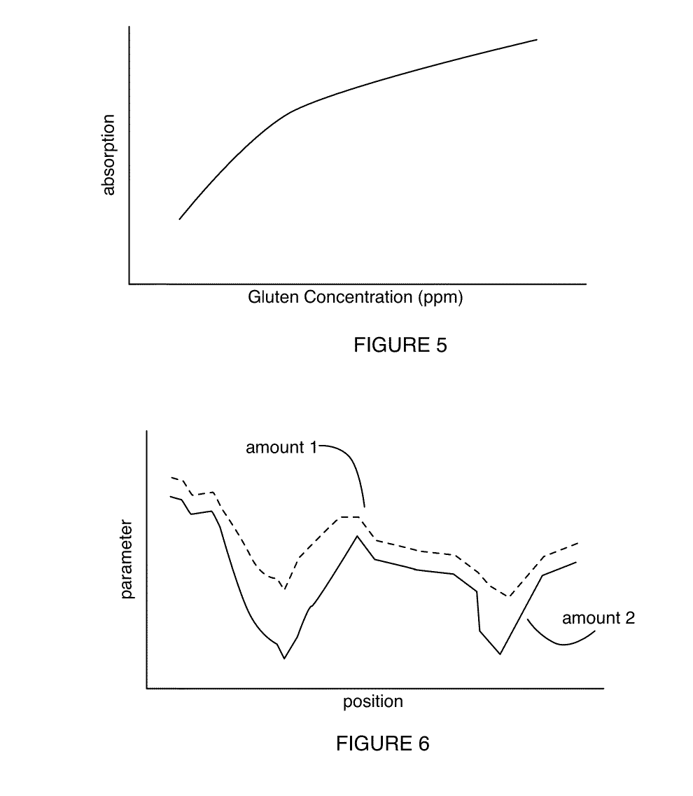

In generating an analysis in Block S160, the analysis preferably includes qualitative and/or quantitative information pertaining to presence (or absence) of the allergen constituent in the consumable sample, and/or an indication of reliability of the results produced at the detection region of the allergen indicator module. In relation to the variation of the detection region comprising a control region, a test region, and a competitive region described above, the analysis can be derived from detection of patterns in activation in the detection region, and output qualitative descriptions of positivity or negativity of the test (e.g., a negative result, a positive result, a high positive result, a very high positive result, etc.). The analysis can additionally or alternatively provide quantitative information regarding an amount (e.g., concentration, volume, mass) of the allergen constituent within the consumable sample. In one variation involving data from a photodiode, generating the analysis in Block S160 can include identifying absorption peaks detected upon illumination of the detection region of a lateral flow element (e.g., over time, taking into account kinetics of a reaction at the detection region), and associating an amount of absorption with an amount (e.g., concentration in parts per million) of an allergen present in the consumable sample, an example of which is shown in FIG. 5.

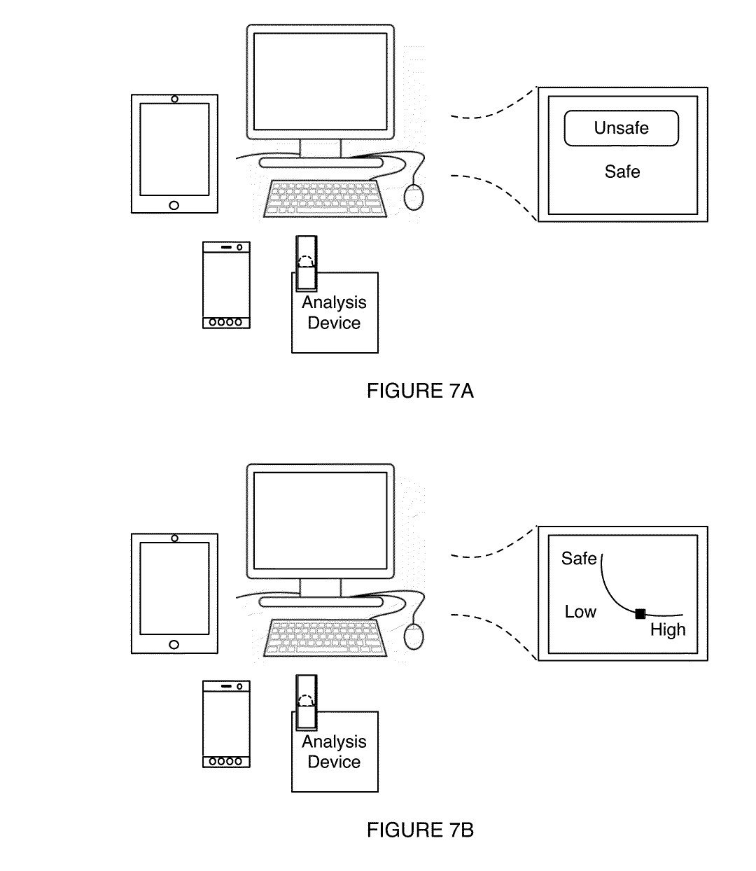

In one variation involving image data from a camera module, generating the analysis in Block S160 can comprise characterizing intensity (e.g., average intensity, peak intensity, relative intensity) across an active region of the detection region of the membrane, and associating an intensity parameter (or other image parameter) with an amount (e.g., concentration in parts per million) of an allergen present in the consumable sample, as shown in FIG. 6. Additionally or alternatively, Block S160 can include processing any other suitable data from a detector module (e.g., photodiode, photomultiplier tube, photon detector, etc.). Additionally or alternatively, Block S160 can comprise filtering excitation wavelengths of light used to excite target components at the detection region, and/or filtering emitted wavelengths of light prior to detection at the detector module, in order to facilitate analyses derived from the detection region (e.g., in relation to fluorescence-based assays). Furthermore, Block S160 can comprise processing signals derived from a detection substrate saturated with a volume of the test sample, and/or generating an analysis in any other suitable manner.

Regarding Block S160, in specific examples of detecting the presence of allergen constituents in a consumable sample, generating the analysis of the indication can include detecting the presence of at least 0.1 ppm for gluten-derived allergen constituents, at least 0.005 ppm for peanut-derived allergen constituents, and at least 0.001 ppm for dairy-derived allergen constituents. However, generating the analysis can include any suitable level of detection.

3.7 Providing Information Derived from the Analysis.

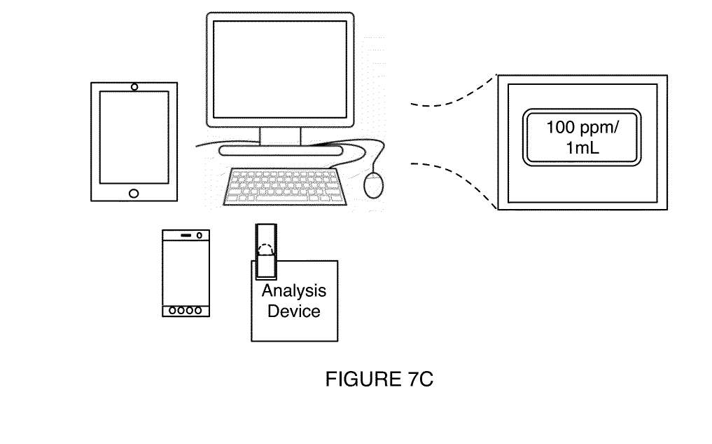

Block S170 recites: providing information derived from the analysis to a user associated with the consumable sample, which functions to inform the user regarding presence, absence, and/or parameters associated with presence of the allergen constituent in the consumable sample. In Block S160, the information can be provided to the user in a visually observable manner (e.g., with text, with rendered images, with rendered video, etc.), in an audibly detectable manner, in a haptically detectable manner (e.g., using a vibration module), and/or in any other suitable manner. In one variation, the information can be provided to the user using a display element (e.g., of an analysis device detecting and processing signals from the allergen indicator module, of a mobile computing device in communication with the analysis device, of a computing device in communication with the analysis device, over a web application accessible using an internet browser, etc.). Additionally or alternatively, in another variation, the information can be provided to the user using a speaker element (e.g., of an analysis device detecting and processing signals from the allergen indicator module, of a mobile computing device in communication with the analysis device, of a computing device in communication with the analysis device, over a web application accessible using an internet browser, etc.). Additionally or alternatively, in another variation, the information can be provided to the user using a vibration element (e.g., of an analysis device detecting and processing signals from the allergen indicator module, of a mobile computing device in communication with the analysis device, of a computing device in communication with the analysis device, over a web application accessible using an internet browser, etc.). Preferably, the information is provided to the user in an intuitive and/or easy to understand manner, such that the user does not need a significant amount of training to understand the information provided to him/her. In one example, as shown in FIG. 7A, the information can be rendered as a message at a display element, wherein the information indicates a binary “Safe/Unsafe” result configured to eliminate potential errors due to user interpretation. In another example, as shown in FIG. 7B, the information can be rendered as a message at a display element, wherein the information qualitatively indicates a severity of presence of the allergen (e.g., along a scale from low presence to extremely high presence) in the consumable sample. Additionally or alternatively, in another example, as shown in FIG. 7C, the information can indicate any suitable type of quantitative information pertaining to an amount/concentration of the allergen in the consumable sample. However, the information can be provided to the user using a combination of the described approaches, and additionally or alternatively be provided to the user in any other suitable manner. In a specific example, providing information derived from the analysis to a user can take 1-10 seconds (e.g., 5 seconds, under 10 seconds, etc.). In another specific example, Block S170 can include providing information derived from the analysis to a user associated with the consumable sample, at a display in communication with the sample analysis device, within two minutes of receiving the consumable sample at the capsule, but can be longer or shorter. In another specific example, the method 100 can comprise initiating a user session upon receiving a consumable sample, and ending the user session upon providing information derived from the analysis to the user. The user session can be completed from start to finish within two minutes, but can be longer or shorter (e.g., from 10 seconds to 10 minutes). In a further specific example, Block S170 can include providing information derived from the analysis to the user within two minutes of receiving the consumable sample. However, Block S170 and/or other Blocks of the method 100 can take any suitable amount of time.

In a variation, Block S170 can include: providing information derived from the analysis to a user device (e.g., a device separate from the analysis device or allergen indicator module) of the user. The user device can be smartphone, smartwatch, tablet, desktop, or any other suitable device. The user device can be associated with any suitable user (e.g., caretaker, consumable sample consumer, consumable sample preparer, manufacturer, retailer, restaurant, etc.). Information is preferably communicated by the device generating the analysis as in Block S160. For example, if an analysis device generates the analysis as in Block S160, the analysis device preferably communicates the information to a user device. However, any suitable device can provide the information to the user device. Information is preferably provided through wireless means (e.g., Bluetooth, WiFi, etc.) but can additionally or alternatively be provided through wired means or other suitable means. Providing the information is preferably in response to generating the information derived from the analysis, but can otherwise be performed at any suitable time. The information is preferably provided as a notification (e.g., a text notification indicating the presence or lack of an allergen constituent in the analyzed consumable sample) at the user device, but can otherwise be presented at the user device.

The method 100 can additionally or alternatively include any other suitable blocks or steps configured to facilitate reception and/or processing of a consumable sample, in order to facilitate detection of the presence of one or more allergens within the consumable sample.

Embodiments of the method 100 and/or system and variations thereof can be embodied and/or implemented at least in part by a machine configured to receive a computer-readable medium storing computer-readable instructions. The instructions are preferably executed by computer-executable components preferably integrated with the system 100 and one or more portions of the processor and/or a controller. The computer-readable medium can be stored on any suitable computer-readable media such as RAMs, ROMs, flash memory, EEPROMs, optical devices (CD or DVD), hard drives, floppy drives, or any suitable device. The computer-executable component is preferably a general or application specific processor, but any suitable dedicated hardware or hardware/firmware combination device can alternatively or additionally execute the instructions.

The FIGURES illustrate the architecture, functionality and operation of possible implementations of systems, methods and computer program products according to preferred embodiments, example configurations, and variations thereof. In this regard, each block in the flowchart or block diagrams may represent a module, segment, or portion of code, which comprises one or more executable instructions for implementing the specified logical function(s). It should also be noted that, in some alternative implementations, the functions noted in the block can occur out of the order noted in the FIGURES. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

As a person skilled in the art will recognize from the previous detailed description and from the figures and claims, modifications and changes can be made to the preferred embodiments of the invention without departing from the scope of this invention defined in the following claims.

We claim:

1. A method for detecting a target substance in a consumable sample, the method consisting essentially of:
receiving the consumable sample at a first chamber of a capsule;
extracting a test sample including an allergen constituent from the consumable sample with an extraction buffer at a second chamber of the capsule, in communication with the first chamber;
upon extraction of the test sample, directly transmitting the test sample from the second chamber, through a valve, and to a sample pad of an immunoassay device in communication with the second chamber;
generating an indication characteristic of presence of the target substance in the consumable sample upon reacting the allergen constituent with a probe at the immunoassay device;
within a sample analysis device surrounding the capsule and including an optical sensor configured proximal the sample pad, generating an analysis of the indication, including at least one of a presence of and an amount of the target substance present in the consumable sample; and
providing information derived from the analysis to a user associated with the consumable sample, at a display in communication with the sample analysis device, within five minutes of receiving the consumable sample at the capsule.

2. The method of claim 1, wherein the probe comprises at least one of: an anti-gliadin antibody; an anti-arachis hypogaea antibody, and an anti-casein antibody.

3. A method for detecting a target substance in a consumable sample, the method comprising:
receiving a consumable sample at a first chamber of a capsule;
extracting a test sample including a first allergen constituent from the consumable sample at a second chamber of the capsule, in communication with the first chamber;
transmitting the test sample to an immunoassay device, in communication with the second chamber;
generating a first indication characteristic of presence of the target substance in the consumable sample upon interfacing the test sample with a probe configured to react with the first allergen constituent at a first testing region of the immunoassay device;
proximal in time to generating the first indication characteristic, generating a second indication characteristic upon interfacing the test sample with a bioconjugate configured to react with the first allergen constituent at a first competitive region of the immunoassay device, wherein the bioconjugate comprises an antibody and a biomolecule with a biomolecule affinity for the antibody less than or equal to the first allergen constituent affinity for the antibody;
generating an analysis of the first and the second indication characteristics, the analysis including at least one of a presence of and an amount of the target substance present in the consumable sample; and
providing information derived from the analysis to a user associated with the consumable sample.

4. The method of claim 3:
wherein the immunoassay device comprises a conjugate pad configured to provide the allergen constituent with a bioactive substrate selected from at least one of: a gold particle and a latex particle, and
wherein the allergen constituent is at least one of: a gluten-derived analyte, a peanut-derived analyte, and a dairy-derived analyte.

5. The method of claim 3, further comprising:
flowing, at the immunoassay device, the test sample in a downstream flow from the testing region, through the competitive region, and to a control region of the immunoassay device;
after generating the second indication characteristic, generating a third indication characteristic upon interfacing the test sample with the control region configured to capture remaining biomolecules of the test sample.

6. The method of claim 5, wherein generating the analysis includes detecting an invalid result upon detecting activation of any of the first indication characteristic and the second indication characteristic, without activation of the third indication characteristic.

7. The method of claim 5, wherein generating the analysis includes detecting a positive result upon detecting activation of the first, the second, and the third indication characteristics

8. The method of claim 5, wherein generating the analysis includes detecting a high positive result upon detecting activation of the first and the third indication characteristics, without activation of the second indication characteristic.

9. The method of claim 5, wherein generating the analysis includes detecting a very high positive result upon detecting activation of the third indication characteristic, without activation of the first and the second characteristics.

10. The method of claim 3, wherein transmitting the test sample comprises, upon extracting the test sample, directly transmitting the sample from the second chamber, through a valve, to the immunoassay device.

11. The method of claim 10, wherein the immunoassay device is a test substrate embedded within the capsule and wherein directly transmitting the sample comprises directly transmitting the sample, through a valve, to a sample pad of the test substrate.

12. The method of claim 3, further comprising:
generating a third indication characteristic upon interfacing the test sample with a second probe configured to react with a second allergen constituent at a second testing region of the immunoassay device;
after generating the third indication characteristic, generating a fourth indication characteristic upon interfacing the test sample with a second bioconjugate at a second competitive region of the immunoassay device,
wherein generating the analysis of the first and the second indication characteristics comprises generating the analysis of the third and the fourth indication characteristics.

13. The method of claim 3, wherein the biomolecule is a second allergen constituent associated with the first allergen constituent.

14. A method for detecting a target substance in a consumable sample, the method comprising:
receiving a consumable sample;
extracting a first test sample including a first target biomolecule from the consumable sample at a chamber of a capsule;
upon extraction of the test sample, directly transmitting the test sample from the chamber, through a valve, and to an immunoassay device in communication with the chamber;
generating a first indication characteristic of presence of the target substance in the consumable sample upon interfacing the first test sample with a probe configured to react with the first target biomolecule at a testing region of the immunoassay device
generating a second indication characteristic upon interfacing the first test sample with a bioconjugate configured to react with the first target biomolecule at a competitive region of the immunoassay device, wherein the bioconjugate comprises a bioconjugate constituent and a biomolecule with a biomolecule affinity for the bioconjugate constituent less than or equal to the first target biomolecule affinity for the bioconjugate constituent;
generating an analysis of the first and the second indication characteristics, the analysis including at least one of a presence of and an amount of the target substance present in the consumable sample; and
providing information derived from the analysis to a user associated with the consumable sample.

15. The method of claim 14, wherein receiving the consumable sample comprises receiving the consumable sample at the chamber of the capsule, and wherein extracting the test sample comprises extracting the test sample by combining the consumable sample with an extraction buffer at the chamber.

16. The method of claim 15, wherein directly transmitting the test sample comprises directly transmitting the test sample through a port of the capsule to the immunoassay device within the capsule.

17. The method of claim 14, further comprising, at the immunoassay device, flowing the test sample through the immunoassay device, wherein the competitive region is downstream the testing region of the immunoassay device.

18. The method of claim 17, wherein generating the analysis includes detecting a negative result upon detecting activation of the first indication characteristic, without activation of the second indication characteristic.

19. The method of claim 17, wherein generating the analysis includes detecting a high positive result upon detecting insufficient activation of the first and the second characteristics.

20. The method of claim 14, wherein the biomolecule and the target biomolecule are allergen constituents selected from at least one of: a gluten-derived biomolecule, a peanut-derived biomolecule, and a dairy-derived biomolecule.

21. The method of claim 14,
wherein extracting the first test sample comprises combining a first portion of the consumable sample with a first extraction buffer,
the method further comprising extracting a second test sample including a second target biomolecule from a second portion of the consumable sample with a second extraction buffer; and
generating a third indication characteristic of presence of the second target biomolecule in the consumable sample upon interfacing the second test sample with a second probe configured to react with the second target biomolecule.
